(19) United States
(12) Patent Application Publication
(10) Pub. No.: US 2006/0122513 A1
(43) Pub. Date: Jun. 8, 2006

(54) Doppler helmet

(76) Inventor: William Gregory Taylor, San Antonio, TX (US)

Correspondence Address: WILLIAM G. TAYLOR, 6612 MANOR HILL RD, SAN ANTONIO, TX, 78257, US

(21) Appl. No.: 11/005,514

(22) Filed: Dec. 6, 2004

(51) Int. Cl. A61B 8/14

(52) U.S. Cl. 600/459

(57) ABSTRACT

A helmet manufactured for one specific person, made from rigid synthetic materials, to specifications determined by data obtained from a previously obtained MRI (magnetic resonance imaging) scan of that person's brain, intracranial arteries, and skull. The helmet and its attached adapters hold in place various Doppler probes directed at specific arteries, both intra-cranial and extra-cranial, to provide continuous readings of the velocity of the blood flow through those arteries.

### CROSS REFERENCE TO RELATED APPLICATIONS

U.S. Patent Documents

| | | | |
|---|---|---|---|
| 4,103,679 | August 1978 | Aronson | 600/456 |
| 4,970,907 | November 1990 | Flynn | 73/866.5 |
| 5,409,010 | April 1995 | Beach | 600/455 |
| 6,694,167 | February 2004 | Fare | 600/424 |
| 6,752,812 | June 2004 | Truwit | 606/130 |
| 6,773,400 | August 2004 | Njemanze | 600/454 |

### OTHER REFERENCES

M. Hennerici, MD, W. Rautenberg, MD, G. Sitzer, MD, and A. Schwartz, MD; Transcranial Doppler ultrasound for the assessment of intracranial aerial flow velocity—Part 1, Examination technique and normal values; Surg Neurol 1987; 27; 439-48

### BACKGROUND OF THE INVENTION

1) Field of the Invention

This invention relates to a device for continuously monitoring the blood flow in specific arteries inside the brain.

2) Description of Prior Art

Many patients, who have suffered a sub-arachnoid hemorrhage from a ruptured intracranial aneurysm, are at risk of developing vasospasm in some of the major intracranial arteries. This vasospasm causes a prolonged, but reversible, narrowing of those arteries, causing a decrease in the blood flow, and subsequently a profound decrease in the blood supply to that part of the brain served by those arteries. Untreated this will produce a stroke and possibly death.

Tans-cranial ultrasound Doppler monitoring of intracranial arteries has been done for several years. The technique was published by Hennerici et. al. in 1987, and is essentially unchanged today. The current technique involves manually applying the Doppler probe over the temporal bone, adjusting its position and direction until a strong signal is obtained, and recording the velocity in the artery at which it is directed. When a Doppler signal is directed at a major artery, in the direction of blood flow, it can detect the velocity of blood flow in that artery. This velocity has a direct relationship to the volume of blood flow and, consequently, to the degree of vasospasm in the artery.

A Doppler signal is also obtained from an external carotid arty, in order to compare intracranial to extra cranial blood flow velocities. This procedure is repeated at various intervals, over several days, during which vasospasm is anticipated.

There are no readily available devices that can maintain a prolonged continuous monitoring of intracranial arterial blood flow that could alert the physician to the onset and course of vasospasm. Most Doppler probes are designed to be hand held devices and used for short time periods. Some devices can clamp such a probe in a fixed position, but are not designed to be secured to the patient's head. There are devices that mount on a patient's head like headphones for continuous monitoring. These, however, are designed for an alert patient who can maintain the probes in proper position, are easily dislodged from their desired position, and are designed for short term use. There are frames that bolt securely to a patient's head, and can accommodate bolt-on apparatuses that can identify the trajectory to major intracranial arteries. These devices are used to guide surgical procedures and, because of their substantial cost, cannot be used by one patient for several days at a time. The same is true for devices that fix a patient's head in a constant position to deliver a dose of radiation to intracranial lesions.

### BRIEF SUMMARY OF THE INVENTION

This application provides for a helmet that is made to exactly conform to a specific person's head. This helmet is made of a rigid plastic material to specifications dictated by the data obtained from a MRI scan previously obtained. The inside topography of the helmet is matched to that of the wearers skull because of the acquired data. This data also records several important anatomical landmarks of the skull which are identified by localizing lines inscribed on the surface of the helmet. The exact position and direction of the blood flow of the major intracranial arteries is determined from the data. This data then identifies the exact points where the vectors of this flow will intersect the helmet. The helmet is constructed with windows at these points, which will become the points of attachment of the monitoring Doppler probes. These Doppler probes are fixed to the helmet and connected to a signal generator-receiver, to continuously monitor signals simultaneously from several intracranial arteries, compute the variance in velocity between extra cranial flow and intracranial flow, and set off an alarm if parameters exceed the pre programmed limits.

### BRIEF DESCRIPTION OF THE SEVERAL VIEWS OF THE DRAWINGS

FIG. 1 is a view of the front of the helmet (1) showing it fitted to the user with a Doppler probe (2) mounted to an adaptor (3), attached over the left temporal bone, and a representation of the left internal carotid artery (4) with left middle cerebral (5) and left anterior cerebral branches (6).

FIG. 2 is a view of the right side of the helmet (1) showing it fitted to the user with a Doppler probe (2) mounted to an adaptor (3), attached over the right temporal bone, and a representation of the right internal carotid artery (7) with the right middle cerebral (8) and right anterior cerebral (9) branches.

FIG. 3 is a view of the front of the helmet (1) fitted to the user with a Doppler probe (2) mounted to an adaptor (3) attached over the apex and a representation of the basilar artery (10) and some of its branches (11).

FIG. 4 is a view of the right side of the helmet (1) fitted to the user with a Doppler probe (2) mounted to an adaptor (3) attached over the apex and a representation of the basilar artery (10) and some of its branches (11).

FIG. 5 is a view of the right side of the helmet (1) fitted to the user with a Doppler probe (2) mounted to an adaptor (12) attached to the lower edge of the helmet and with a representation of the external carotid artery (13).

FIG. 6 is a view of the right side of the helmet (1) fitted to the user showing the orientation lines (14) inscribed on the helmet directed at anatomical landmarks.

FIG. 7 is a typical Doppler probe (2).

FIG. 8 is a Doppler probe (2) secured to an adapter (3) that attaches to the helmet (1).

FIG. 9 is a Doppler probe (2) secured to the adapter (12) that attaches to the helmet (1) over the external carotid artery (13).

### DETAILED DESCRIPTION OF THE INVENTION

The object of this invention is to provide a means to maintain a Doppler probe in an exact position, with respect to a patient's intracranial arteries, to continuously monitor blood flow in an those arteries.

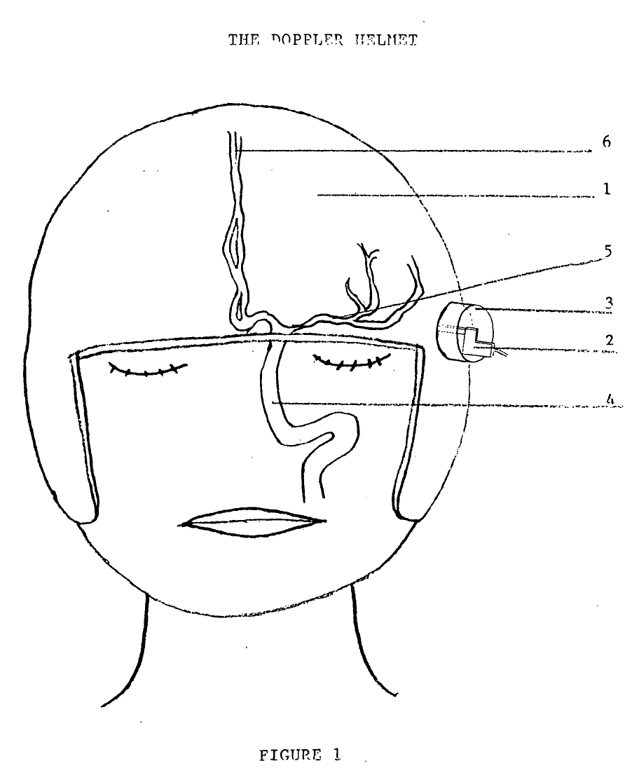

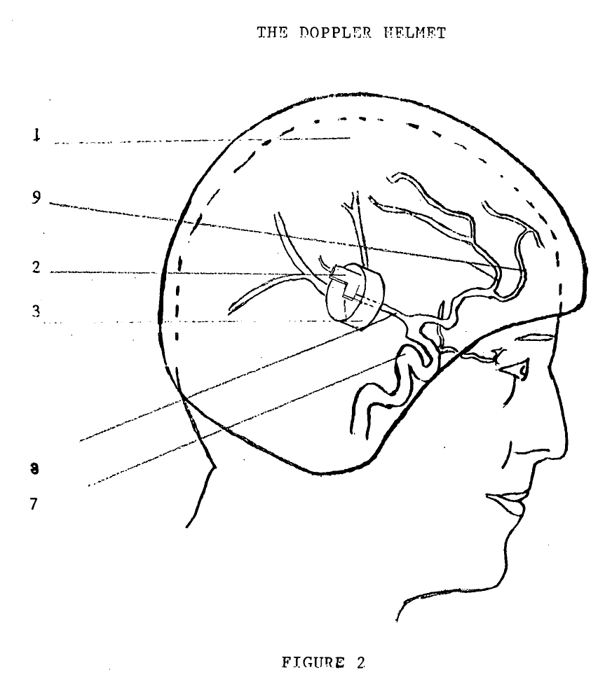

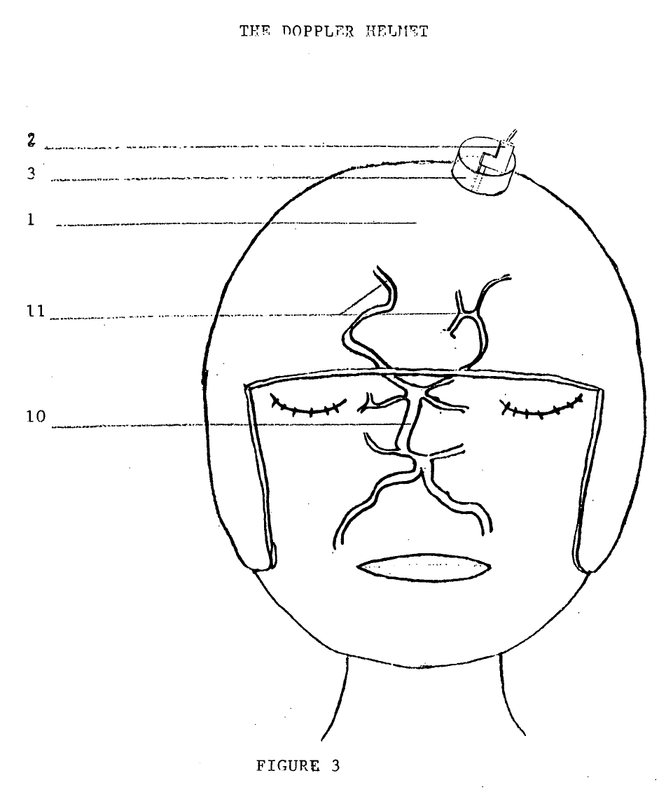

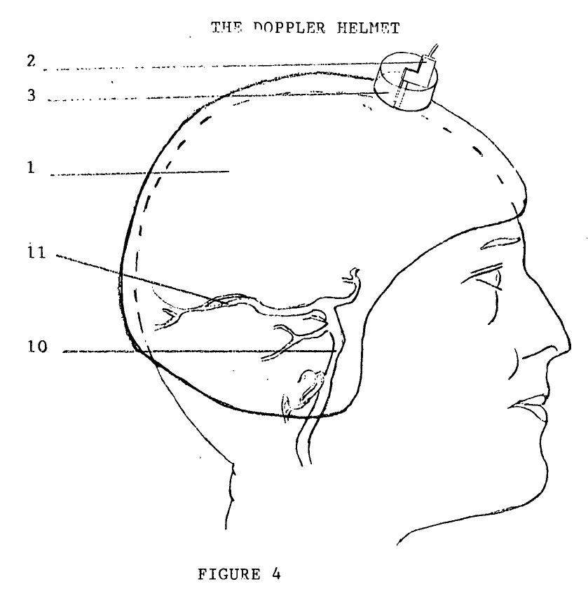

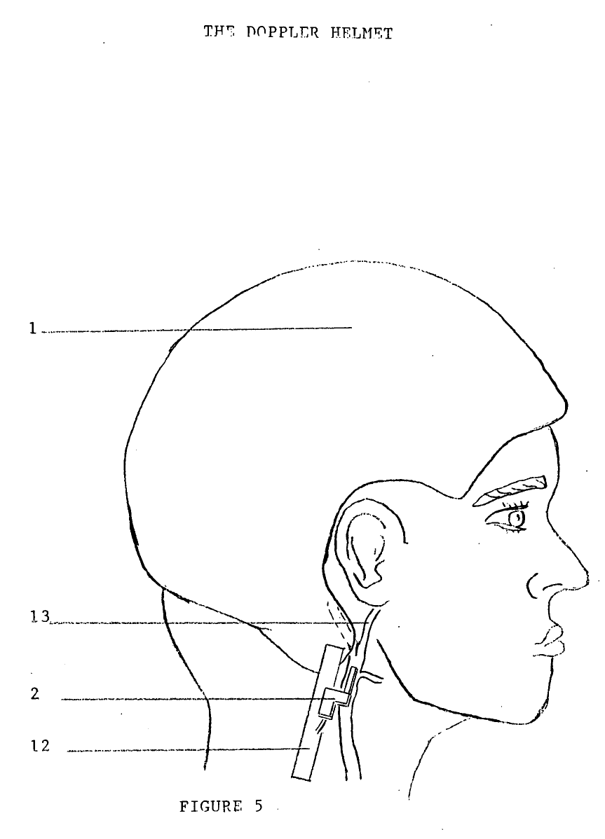

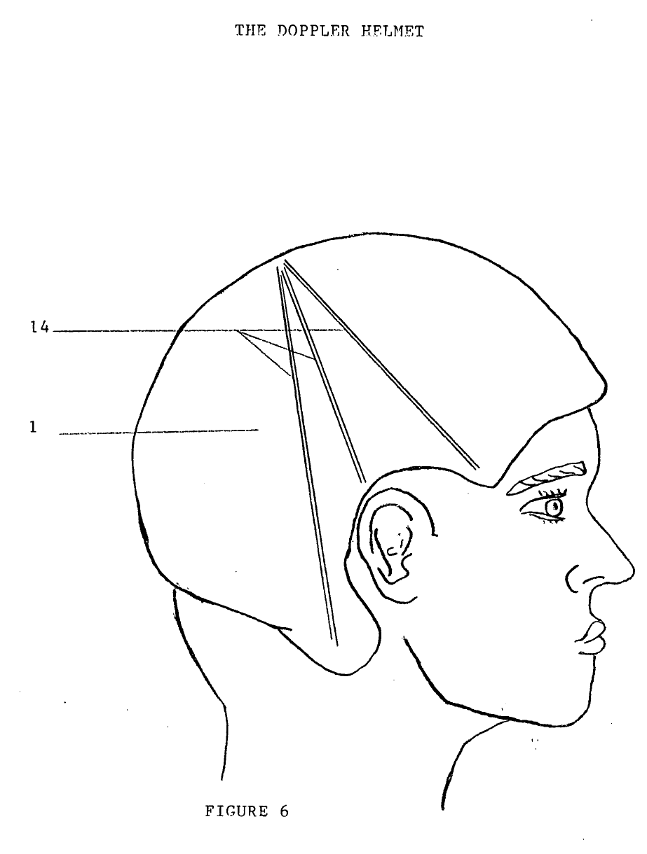

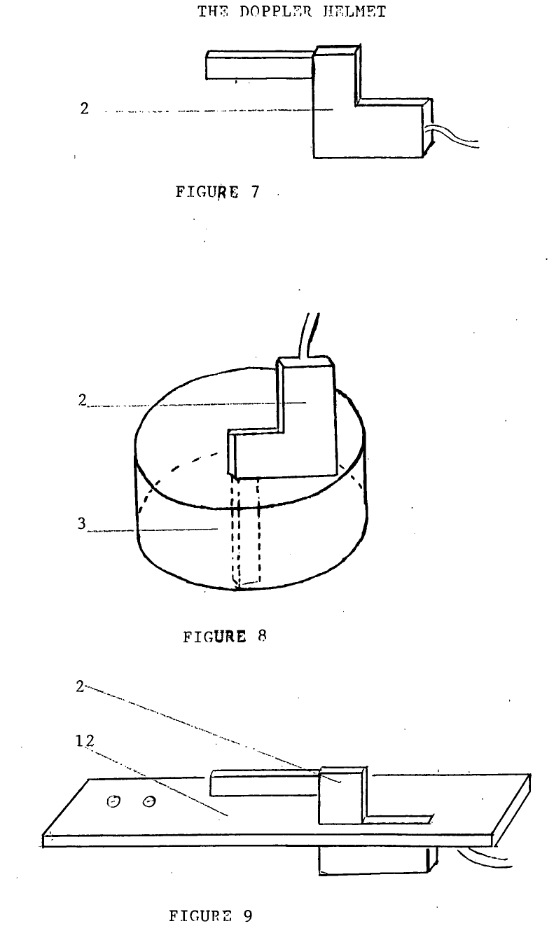

With reference to FIGS. 1-9, it is seen that the helmet (1) is made to fit on the head of one specific person, and the helmet (1) is fitted with several adapters (3) (12) that hold Doppler probes (2) secured to predetermined windows on the helmet. These adaptors can be adjusted in two planes and locked in position such that the directional orientation of each Doppler probe faces directly at the vector of blood flow in the artery it is monitoring.

In current use is the machinery to produce accurate physical models of a patient's skull and intra-cranial arteries derived from the data obtained with medical imaging studies, i.e. MRI. Once the raw data is obtained, specialized software is used to construct a virtual 3D model. This is fed to a prototyping machine that produces a final model in physical form. This same technology and machine can also be used to create a shell (helmet) with the exact shape of a patient's skull. The virtual model will also identify the exact spatial relationship between the major intra-cranial arteries and the skull, and therefore, the helmet. This allows the prototyping machine to make a helmet with pre cut windows exactly in line with the flow vectors of the major intracranial arteries.

The same data can provide coordinates of several skull landmarks that are readily identifiable on a patient, i.e. external auditory canal, nasion, zygoma, mastoid process. When the virtual model of the helmet is made the software can identify these landmarks with respect to an arbitrary point on the helmet surface. When the physical model is made if can be inscribed with surface lines oriented from that arbitrary point toward the landmarks. This will help insure that the helmet is fitted to the patient properly.

The helmet is then fitted with special mounting adaptors, designed to secure to the windows while holding a probe pointing at the arteries. The adaptor is further designed to be mobile in two planes for fine adjustment of probe direction; and designed to be able to be locked in position once that direction is identified. This sets the probe is in position for continuous monitoring.

Because the original data identifies all the major intracranial arteries, and their flow vectors, the helmet provides the opportunity to monitor several major arteries simultaneously with different probes. It is also seen, from FIG. 5, that the left or right external carotid artery can also be monitored with a properly mounted and directed probe.

When a patient suffers a subarachnoid hemorrhage (SAH), he is at risk of developing vasospasm of some of the major intracranial arteries at some time during the first two weeks after the bleed. When vasospasm occurs, the muscle wall of the artery contracts, narrowing the lumen, and restricting the blood flow. Prolonged vasospasm will cause a stroke in that part of the brain relying on this artery. The current management of vasospasm is somewhat risky, and is not initiated in the absence of true vasospasm; but should be initiated as soon as it is detected. Whereas current practice involves intermittent manual monitoring, the helmet can be set up for automatic constant monitoring.

It has been found to be helpful to monitoring the extra-cranial blood flow as well. Those physiological factors that influence heart rate and blood flow generally effect both intra-cranial and a extra-cranial arteries similarly. Vasospasm from SAH does not affect the extra-cranial arteries. Because of this, the rise in the ratio of flow in the intra-cranial artery to the flow in the extra-cranial artery is the best indication of active vasospasm. The helmet allows for monitoring both. The monitor can be easily programmed to constantly calculate and read out that ratio, and to alarm at a pre determined level and even to print a paper copy of several minuets of calculations.

Although the invention has been described in connection with the preferred embodiment, it is not intended to limit the invention's particular form set forth, but on the contrary, it is intended to cover such alterations, modifications, and equivalences that may be included in the spirit and scope of the invention as defined by the appended claims.

I claim:

1. A helmet having an inner surface, and an outer surface, the helmet includes multiple holes, or ports, specifically located by reference to data obtained from a computerized tomographic scan of a user's head; with adapters fitted to these ports that secure Doppler probes positioned to face specific major arteries inside and outside of the brain of the user, and aligned to the flow of blood in those arteries directly toward or away form the probes,

2. The helmet according to claim 1 wherein the helmet is also fitted with an adaptor that holds a Doppler probe over an external carotid artery oriented to monitor the blood flow in that artery,

3. The helmet according to claim 1 wherein the position of the ports and adapters as well as the directional settings of the probes has been determined from data obtained from a computerized scan of the users head,

4. The helmet according to claim 1, wherein the helmet is made of a rigid plastic-like substance by computer controlled machinery using the data obtained from a computerized scan of the user's skull and major intracranial arteries,

5. The helmet according to claim 1, wherein the outer surface of the helmet is inscribed with lines when extrapolated over the user's head, intersect on specific landmarks of the user's head and skull,

6. The helmet According to claim 1, wherein the inner surface of the helmet is a complex topographic shape constructed from data obtained by a computerized scan and made to conform closely to the head of the user.
